(19) United States
(12) Patent Application Publication
(10) Pub. No.: US 2024/0245630 A1
(43) Pub. Date: Jul. 25, 2024

(54) DUAL-PURPOSE ANALGESIC COSMETIC COMPOSITION

(71) Applicant: Coverology Inc., Scottsdale, AZ (US)

(72) Inventors: Alexandra Evelyn Mora, Phoenix, AZ (US); Fadi Mourad, Redondo Beach, CA (US)

(21) Appl. No.: 18/408,745

(22) Filed: Jan. 10, 2024

Related U.S. Application Data
(60) Provisional application No. 63/440,551, filed on Jan. 23, 2023.

(51) Int. Cl. A61K 31/125; A61K 8/19; A61K 8/29; A61K 9/00; A61Q 1/04

(52) U.S. Cl. CPC A61K 31/125; A61K 8/19; A61K 8/29; A61K 9/0014; A61Q 1/04

(57) ABSTRACT

Novel dual use compositions for the obscuring and soothing of tender pigment imperfections are disclosed. These pre-mixed compositions represent a significant aesthetic improvement over the prior art, as well as exhibiting increased resilience over said prior art. In addition to their cosmetic properties, said novel compositions possess and exhibit analgesic properties, and include a topical analgesic for addressing cold sores, a waxy ingredient, a colorant or pigment, and a stabilizer.

### CROSS-REFERENCE TO RELATED APPLICATIONS

This application claims priority to U.S. Provisional Patent Application No. 63/440,551, filed on Jan. 23, 2023. The entire disclosure of this application is hereby incorporated by reference in its entirety.

### FEDERALLY SPONSORED RESEARCH OR DEVELOPMENT

[Not Applicable]

### BACKGROUND OF THE APPLICATION

In 2016, Americans spent over fifty-five billion dollars on cosmetics. Much of this went to a growing industry segment known as camouflage cosmetics. Often referred to as concealers, this segment, year over year, since at least 2011, has seen substantial growth, accounting for 30.5 million dollars in profit in 2011 alone.

At the same time, the sale and development of dermatologic or over the counter formulations intended to alleviate or limit epidermal pain or discomfort or pain, has exploded. One recent homeopathic article alone explored the efficacy of potato paste, cornstarch, baking soda, aloe, vinegar, oatmeal, and vinegar, in relieving pain associated with burns, acne, and a host of additional skin imperfections.

An additional epidermal imperfection, which the pharmaceutical/homeopathic industries have prolifically sought to soothe, is Herpes Simplex-1 (HSV). HSV outbreaks involve swollen and discolored facial blemishes or lesions commonly referred to as cold sores or fever blisters, which form either labially or in the facial region immediately surrounding the lips (the facial labial or perioral region). Despite this prolificness, however, the number of dual-use compositions the homeopathic/pharmaceutical industry has produced in conjunction with the cosmetics industry, which both conceals cold sores and eases the discomfort they cause, could be counted on less than one hand. And none of these scant offerings are desirable.

This failure of the cosmetics and dermatologic/homeopathic industries to create a desirable dual-use composition that functions as both a cosmetic and an analgesic has left a glaring market hole.

Cold sores or fever blisters are far more common than most suspect, with upwards of 90% of Americans, as of 2015, having been exposed to HSV. HSV infection is ubiquitous, with 600,000 new cases developing each year. Further, approximately one third of patients who experience the initial HSV lesion undergo lifelong recurrences. An unscientific inkling of the absence of a dual-use composition described above can be gleaned with a simple YouTube® search of, “hiding cold sores,” which reveals page after page of suggested means of applying cosmetics over compositions intended to soothe the lesions, rather than a single product intended to both soothe and conceal.

However, despite these parallel increases in both sales/revenue and demand, the hole remains. With regard to HSV lesions, the labial region of the face is fairly inhospitable. Any product attempting to either conceal, cover, and otherwise color or soothe lesions found thereabout must be impervious to perspiration, saliva, skin oils, and erosion precipitated by the human propensity to frequently touch the region about the mouth. Furthermore, any products meant for this region must be safely ingestible, as their proximity to the mouth will inevitably lead to some, even if miniscule, oral consumption. Lastly, any such product must combine a cosmetic pigment with a soothing agent in a stable, non-reactive manner. Accomplishing all of this including creating a composition that functions just as well in epidermal regions geographically removed from the lips, is a daunting task.

Attempts to address the issue have not been successful. A variety of companies, such as ABREVA®, which have sold cold sore medications, produced some version of a clear patch. A cold sore sufferer adhesively applied said patch to the lip or perioral/labial region in contact with the blister and thereafter, may apply a concealer to the outer side of the patch. These patches, however, did a poor job of concealment. They are apt to peel away and are easily detectable, even after being covered with a cosmetic, as the human eye quickly detects a difference between the texture of skin and that of the patch's surface. For its part, the cosmetics industry has produced a myriad of web and magazine articles on how to apply a combination of concealer and soothing salve to fever blisters. These articles, however, also do a poor job. Their proffered solutions suggest a combination of two products, a concealer and a cold sore pharmaceutical, which are chemically incompatible. For example, the competing hydrophilic and hydrophobic properties of the components prevent uniform application. The attempted application of cosmetics stacked over medicaments result in inadequate application or even removal of either or both the medicament or the cosmetic (e.g., by sliding or smearing off or inability of the colorized pigment to adhere to the lip and/or surrounding region). Their combination results in gloppy, runny, and all around ineffective coverage. Additionally, even if said articles taught sound concealment/soothing advice, those who follow the articles' advice would be required to purchase a minimum of two separate products, which is expensive.

One barrier to the creation of products that conceal facial blemishes is pain. Further, facial blemishes often hurt. Cold sores, in particular, are painful. Tactile contact with painful bodily regions, from broken bones to acne, is unpleasant. This is doubly true when that contact may involve prolonged rubbing and friction, as both cosmetic composition and therapeutic composition application does. The present application, however, offers a solution. The present invention teaches a dual-use composition that reduces pain or discomfort associated with cold sores upon the composition's application. More specifically, the present composition facilitates said rapid pain reduction by incorporating a cold sore analgesic such as camphor into its composition. For a variety of reasons highlighted below, however, prior art cosmetic compositions utilizing camphor in accordance with the present invention, despite its ability to soothe painful epidermal irritation, are conspicuously absent. In concentrations of, e.g., between 3% and 11%, camphor immediately interacts with nerve endings to not only relieve pain, but to foster a pleasurable sensation as well.

Prior art compositions offer unsatisfactory solutions. For instance, U.S. Pat. No. 4,822,601 discloses a cosmetic base exhibiting therapeutic properties in the sense of, e.g., conditioning the skin and wound coverage. However, the composition this patent teaches would be an inadequate epidermal and cold sore soother and concealer. It does not resolve the need for a composition that serves as a cosmetic and fast-acting analgesic for treatment of cold sores. Similarly, another composition that was marketed under the brand CoverAid™ does not incorporate a compound for which the primary function is pain relief for treatment of cold sores. Further, its formulation is far too thick to effectively conceal imperfections located elsewhere on the epidermis, including numerous facial regions.

The present application is harmoniously compatible with the increasingly common belief that the human body, where possible, should be allowed to heal itself and that synthetic pharmaceuticals often create more problems than they solve. For this reason, preferred embodiments of the present invention serve to provide cover, concealment, and analgesic relief using natural cold sore analgesics. In addition, certain alternative embodiments of the present invention may add to the inventive composition natural or man-made ingredients that may assist in wound healing or may mitigate aspects of infection by inhibiting viral replication, preventing bacterial growth, or otherwise providing an environment that reduces virulence of an infective agent.

Accordingly, the present application solves the need for a dual-use composition for topical application including to the lips and/or surrounding tissue that reduces pain and discomfort once applied using a medicament or analgesic that acts to treat cold sores and/or HSV and also serves as an aesthetically-pleasing cosmetic and concealer.

### SUMMARY

The present application overcomes problems with prior art compositions and generally relates to a composition for topical application including to the lip and/or labial region of the face comprised of at least one cold sore analgesic component and at least one cosmetic component such as a colorant or pigment. The present invention surprisingly is able to act as a cold sore analgesic that acts quickly to reduce pain and/or discomfort associated with cold sores (also referred to as fever blisters or HSV outbreaks) while simultaneously serving as a concealing, aesthetically-pleasing colorized cosmetic that allows for easy application by the user. In a preferred embodiment, the composition is substantially hydrophobic or anhydrous, for example, oil-based rather than hydrophilic or water-based. In such embodiments, the composition is water proof providing a significant advantage to the user. In other embodiments, where the cold sore analgesic is hydrophilic, the composition has hydrophobic and hydrophilic components. The dual use composition is thereafter stackable allowing for additional application of cosmetics by the user if desired. These and other advantages and novel features of the present invention, as well as details of illustrated embodiments thereof will be more fully understood from the following description.

It is an object of the present disclosure to provide a dual use cosmetic composition comprised of at least one analgesic, such as camphor. The analgesic is a cold sore analgesic known to act quickly to reduce the pain, inflammation, and/or discomfort associated with cold sores (fever blisters/HSV outbreaks) and/or act to heal them in whole or in part.

It is another object of the present disclosure to provide a dual use cosmetic composition comprised of at least one waxy ingredient, such as beeswax, that acts as a substantially hydrophobic component to control the rigidity or thickness of the cosmetic.

It is yet another object of the present disclosure to provide a dual use cosmetic composition comprised of at least one stabilizer or stabilizing agent (which can also act as an emulsifier as needed), such as cetearyl alcohol, which, in conjunction with a solubilizer, such as Meristant (hydrogenated methyl abietate). This component provides structural stabilization for the composition and, if needed, ensures that the present composition mixes and remains mixed, eliminating the persistent prior art drawback of separating into layers.

It is another object of the present disclosure to provide a dual-use composition that contains cosmetic coloring or pigmentation as well as a pigment base such as mica or titanium dioxide.

It is another object of the present disclosure for the composition to include other optional components such as texture modifiers, conditioning agents, or other composition enhancements.

It is another object of the present disclosure to provide a dual use cosmetic composition that is both a concealer and an analgesic, which can be in the form of a liquid, cream, ointment, gel, powder, or a pressed powder. The composition may be packaged in an applicator or other package for convenient self-application by a user.

In one embodiment of the present application, the dual use cosmetic and analgesic composition for topical application to the lip and/or labial region may comprise at least one cold sore analgesic, at least one pigment, at least one waxy ingredient, and at least one stabilizing agent such as an emulsifying agent, wherein the composition is substantially hydrophobic. In another aspect, the present application relates to a dual use cosmetic and analgesic composition for topical application to the lip or labial region comprising at least one cold sore analgesic, at least one pigment, at least one waxy ingredient, and at least one emulsifying agent, wherein the cold sore analgesic is substantially hydrophilic. In one aspect, the composition is configured for topical application to the lip and/or labial region of the face.

In one aspect, the present application relates to a cosmetic application apparatus comprising a container comprising the dual use cosmetic and analgesic composition and at least one applicator for topical application of the composition.

In another embodiment, the present application relates to a kit comprising a container comprising a housing containing a dual use cosmetic and analgesic composition, and at least one applicator for topical application of the composition, wherein the composition comprises at least one cold sore analgesic, at least one pigment, at least one waxy ingredient, and at least one stabilizing agent. The kit may include a plurality of applicators for topical application of the composition.

In one embodiment, the cold sore analgesic is a pain reliever for treatment of cold sores. In an embodiment, the cold sore analgesic comprises camphor and/or phenol. In another embodiment, the cold sore analgesic is camphorated phenol and may be present in an amount of about 3 to 11% by weight of the composition.

In one embodiment, the pigment comprises iron oxide. In yet another embodiment, the pigment base comprises titanium dioxide. In one embodiment, The pigment may comprise iron oxide in amount between about 0.1 to 5% by weight. The pigment and pigment base together may comprise about 1 to 10% by weight of the composition.

In one embodiment, the dual use cosmetic and analgesic composition for topical application to the lip and/or labial region may further comprise a stabilizing agent that is a solubilizer and the solubilizer is hydrogenated methyl abietate. The stabilizing agent may be an emulsifying agent such as cetearyl alcohol.

In other embodiments, the composition may further contain any of at least one pigment base, at least one texture agent, at least one conditioning agent, and/or other additives that further enhance the composition.

In one embodiment of the present application, the dual use cosmetic and analgesic composition is water proof.

In another embodiment, the present application relates to a method of manufacturing a dual use cosmetic and analgesic composition for topical application comprising preparing a first premix by mixing a cold sore analgesic with a stabilizing agent; preparing a second premix by mixing at least one waxy ingredient with another ingredient; preparing a third premix comprising a coloring agent; preparing a fourth premix by mixing the second premix and third premix; and combining the fourth premix with the first premix and a homogenizer. The first premix may be prepared at a temperature of about above 45 C. In an embodiment, the step of combining the fourth premix with the first premix and homogenizer is prepared at a temperature of about above 40 C.

### DETAILED DESCRIPTION

The following technical disclosure is exemplary and explanatory only and is not necessarily restrictive of the invention as claimed.

The present disclosure provides a novel dual use composition for topical application comprising at least one cold sore analgesic, at least one cosmetic colorant or pigment and may include at least one pigment base, at least one hydrophobic waxy ingredient, and at least one stabilizing agent that may be an emulsifier. The waxy ingredient is resistant to phase separation. The dual use composition is formulated so that it may serve as both an aesthetically-pleasing cosmetic such as concealer and/or an analgesic for, e.g., fast-acting pain relief associated with cold sores. In one embodiment, the composition is anhydrous and hydrophobic and has a seamless aesthetically-pleasing consistency such that it can be used as a concealer. The dual-use compositions are intended to both camouflage or conceal visible skin/epidermal imperfections due to cold sores, fever blisters, and/or HSV outbreaks and to case discomfort associated with those imperfections. Additional components may be optionally included to enhance the analgesic or cosmetic properties of the composition and enhance topical application to the lip and perioral/labial region including solubilizers, texturizing agents, conditioning agents, sunscreens, and/or emollients.

Preferably, the ingredients are combined to form a hydrophobic or anhydrous smooth cream. In other embodiments, the ingredients are combined to form a hydrophilic cream, wherein a hydrophilic cream is defined as an emulsion comprised of, but not limited to, oil and water in approximately equal proportions. In alternate embodiments, the composition's ingredients and ratios, may be modified such that the consistency of the composition may be a powder, liquid, gel, ointment, paste, or pressed powder.

The dual use composition of the present disclosure includes at least one cold sore analgesic that is effective via topical application. The cold sore analgesic of the present application may be any skin-safe medicament or other topically-applied compound or chemical composition that acts to quickly reduce or eliminate pain, inflammation, or discomfort associated with cold sores, fever blisters, and/or HSV when applied topically to the lips and/or surrounding labial region. In a preferred embodiment, the cold sore analgesic is a compound that the Food and Drug Administration (the FDA) does not classify as a prescription drug product. The cold sore analgesic is safe and generally non-toxic when applied by the user in appropriate quantities to the user's lip and surrounding area (including accounting for standard potential for ingestion).

One preferred example of such cold sore analgesic includes camphor including camphor USP powder and phenol. The term camphor refers to, e.g., a natural terpenoid found in the wood of the camphor laurel tree of Southeast Asia. It is hydrophobic in nature and is a white, colorless, and crystalline compound at room temperature. In addition to its analgesic nature, it also has antiseptic, and counter-irritant properties. It is used topically and is represented by the formula C₁₀H₁₆O.

As discussed further herein, in a preferred embodiment, the cold sore analgesic is a combination of camphor and phenol, combined to create camphorated phenol. Phenol (carbolic acid, C₆H₅OH), in turn, is the simplest of a group of organic molecules (“phenols”), which are characterized by having a hydroxyl group covalently bonded to an aromatic ring. Phenol is a volatile organic molecule, which is a white crystalline solid at room temperature. It is also, however, somewhat hydrophilic, due to the hydroxyl group on the aromatic ring. It, too has some analgesic properties. Additionally, it possesses some antiseptic properties, as it triggers leakage of cytoplasmic membranes. This antiseptic property is notably efficacious as the present invention seeks to promote the body's own capacity to heal during its period of concealment, a capacity simplified with the antiseptic elimination of negative foreign irritants.

Other analgesics may be used. For example, some FDA-approved pain relievers that may be used as cold sore analgesics in various implementations of the instant composition may include one or more of benzocaine, benzyl alcohol, dibucaine, diphenhydramine, docosanol, dyclonine, hydrocortisone, juniper tar, lidocaine, menthol, phenol, pramoxine, resorcinol, benzyl alcohol, arnica 3×HPUS, and tetracaine. In alternate embodiments, the cold sore analgesic could be salicylic acid or any other topically-effective and skin-safe component with anesthetic properties that can be applied topically to quickly alleviate pain and discomfort associated with cold sores, fever blisters, and/or HSV outbreaks on the lip and labial region of the face. These analgesics may be used either alone or in combination and may be in powder or liquid form. For anhydrous compositions, the analgesic is in a form that binds to or can be incorporated with a colorant is preferred.

The analgesic may be present in the composition in any amount intended to have the desired effect when applied topically to the lip while maintaining user safety including an amount equal to about 3 to 11%, preferably the amount is equal to about 5 to 10%. In one preferred embodiment, camphor and phenol are present each in an amount equal to about 2 to 5% by weight.

The dual use composition of the present disclosure further includes at least one cosmetic agent comprising a colorant or pigment. The colorant or pigment serves as a cosmetic concealer, camouflaging agent, or cosmetic component that enhances the aesthetics of the composition for the user, allowing the composition to be a single source composition for both cosmetic and analgesic purposes. In one embodiment, the colorant or pigment includes at least one metal oxide such as yellow, red, and/or black iron oxide (either alone or in combination). Additional examples of appropriate colorants include brown and bronze iron oxides.

A suitable amount of the colorant or pigment should be included to ensure that the dual use composition achieves the desired aesthetic effect. For example, different types or amounts of cosmetic agent may be included to account for different skin tones, e.g., light, medium, and dark. For example, the pigment may be present in the composition in an amount equal to about 0.1 to 5% by weight. In a preferred embodiment of a light tone formulation, yellow iron oxide is present in an amount equal to about 1 to 5%, preferably 2 to 3% by weight, red iron oxide is present in amount equal to about 0.5 to 5%, preferably 1 to 2% by weight, and black iron oxide is present in amount equal to about 0.1 to 4% by weight and preferably less than 1%. In medium and dark tone formulations, more or less yellow iron oxide may be added (e.g., about 2 or 3%) and red and black iron oxides are present in higher percentages, e.g., about 3 to 4% or 2 to 3%, respectively.

The pigment or coloring agent is dispersed, preferably homogenously, within at least one pigment base. The pigment base and pigment may be premixed in a preferred formulation. In some embodiments, the pigment base may include mica, titanium dioxide, silica, diisostearyl malate, and isopropyl titanium triisosterate. For cosmetic purposes, in a preferred embodiment, the base pigment has a white undertone. The pigment base may be present in an amount suitable to allow for dispersion of the colorant and appropriate aesthetic effect. In one embodiment, the pigment base is titanium dioxide and is present in amount less than about 10% by weight such as between about 1 to 10%, or 1, 2, 3, 4, 5, 6, 7, or 8% by weight of the composition. The amount of pigment base may vary depending on the amount of pigment. For example, the total amount of colorant/pigment combined with pigment base may constitute between about 10 to 15% of the total weight of the composition (preferably about 11 or 12%). In some embodiments, for light tone formulations, there may be more pigment base as compared to colorant (e.g., about 2:1 to 1:5:1). In dark tone formulations, the ratio of pigment base to colorant may be much higher, e.g., about 4:1 or 4.5:1). In other formulations, the ratio of pigment base to colorant may be nearly equal.

The dual use composition of the present disclosure further includes at least one waxy ingredient, which preferably is characterized by hydrophobic or anhydrous properties or in other words is substantially hydrophobic. The waxy ingredient serves as the base component for the composition and allows for smooth application of the dual use composition to the lip and/or labial region. The waxy ingredient may also serve as a skin conditioner. A feature of the waxy ingredient is its non-aqueous and hydrophobic (or even anhydrous) nature that allows for incorporation of other components of the topical composition. In one embodiment, the waxy ingredient may be shea butter, beeswax, sunflower seed wax, flower wax, polyglycerin such as polyglycerin-3, lanolin, cocoa butter, carnauba wax, synthetic wax, paraffin, microcrystalline wax, white petrolatum, and/or any combination thereof.

The waxy ingredient or ingredients may comprise the largest constituent of the composition. For example, the waxy ingredient may account for 20 to 40% by weight composition of the composition. In a preferred embodiment, two waxy ingredients (e.g., beeswax and shea butter) are about 30 to 40% or 30 to 35% weight of the total composition.

The dual use composition of the present disclosure further includes at least one stabilizer or stabilizing agent, which may be an emulsifier or solubilizer. The stabilizer or stabilizing agent provides structure for the composition, for example by enhancing viscosity, increasing density, enhancing the uniformity of product, improving handling properties, prevention of crystallization, or encapsulating molecules, and allows for stable incorporation of the various components of the composition including by emulsification if needed. The stabilizer or stabilizing agent further allows for the composition to serve as both an analgesic and an aesthetically-pleasing cosmetic.

In some embodiments, the stabilizing agent may be a fatty acid such as cetearyl alcohol, Meristant 101L or hydrogenated methyl abictate, polyglyceryl-4 isostearate, or Corum 5014, acrylates copolymer, microcrystalline cellulose, and sodium polyacrylate, and/or combinations thereof.

In one embodiment, beeswax is the preferred waxy ingredient. Unlike lanolin or some of the other lipids often used in lip balms or other cosmetics, beeswax is comprised of varying chain lengths, alcohols, and esters. These long chain components are mostly hydrophobic in nature.

Use of beeswax may necessitate a stabilizing agent that is an emulsifying agent such as cetearyl alcohol. Emulsifiers are preferred when the cold sore analgesic or other components are hydrophilic in nature, particularly substantially hydrophilic. Cetearyl alcohol is a mixture of long chain alcohols and is derived from vegetables. It is largely hydrophobic, with a long hydrophobic “tail.” However, it also has a hydrophilic “head group” from the hydroxyl functional group (—OH). An emulsifying agent, such as cetearyl alcohol, may form a micellelike structure around the immiscible molecules (e.g., camphor, phenol, and pigments) to ensure they remain in suspension within the “solvent” (e.g., beeswax). Additionally, cetearyl alcohol's dual hydrophilic/hydrophobic nature allows it to interact with both, by way of example, the hydrophobic camphor, and the beeswax (head groups in the cetearyl alcohol can interact with the hydroxyl groups found in beeswax). Similar interactions will ensure that the metal oxide pigments remain and do not separate into discrete layers. Alternate embodiments of the present disclosure may include alternate stabilizing agents including emulsifying agents, such as any of those compounds classifiable as polysorbates, if a composition comprising hydrophilic components is preferred. Specifically, for cold sore analgesics that are substantially hydrophilic in nature, an emulsifying agent is preferably used. An appropriate amount of emulsifying agent is used to ensure that the analgesic or other hydrophilic components remain substantially homogenously dispersed within the composition.

The amount of stabilizer in the composition may vary depending, for example, on the nature of the analgesic, level and mechanism of action of the active ingredients, and the level and amount of pigment. For example, in one preferred embodiment, 8 to 15% by weight stabilizer is selected, e.g., 10 or 11% Meristant 101L.

The dual use composition preferably optionally further comprises at least one, and preferably more than one, skin conditioning agent or emollient. The skin conditioning agent or emollient enhances the ability of the composition to soften, moisturize, soothe, and protect the skin. The waxy ingredient may itself serve as a skin conditioning agent, e.g., beeswax, shea butter, cocoa butter, and others may be included to achieve desirable aesthetic and moisturizing properties for the user. As another example, ACTICIRE® from Gattefosse is based on Jojoba, Mimosa, and Sunflower waxes and acts as an emollient, bringing softness to the skin, and as an active, repairing, protecting, and moisturizing dry, damaged skin. Other examples of conditioning agents or emollients will be known to persons of ordinary skill in the art and could include Corum 5014 or lauryl/myristil benzoate, Meristant 101L or hydrogenated methyl abietate, Liponate GC or caprylic/capric triglyceride, aloe, lemon balm, honey, cosmetic petroleum, mineral oil, allantoin, lycine, jojoba esters, sunflower seed wax, polyglycerin, lauroyl lysine, and/or Abil WI 09, polyceryl-4-isotearate, hexyl laurate, dimethicone, glycerin, olive extract (oleuropein) 3×HPUS, alone or in combination. Zinc oxide, zinc, propolis, rhubarb, sage, and licorice root may also act as skin conditioning agents as well as having soothing or therapeutic effects. In a preferred embodiment the skin conditioning agent or emollient ingredient may account for about 10 to 20% by weight composition of the composition.

Those of skill in the art appreciate that additional components may be added to the composition to achieve the desired purpose. For example, therapeutic agents may be added to the compound to provide other benefits, such as supporting healthy healing of the affected area. Additional components may include, for instance, one or more of an anti-viral agent, an antibiotic agent, a microbicide, docosanol 10%, L-Lysine, benzalkonium chloride, colloidal silver, lemongrass 2×HPUS, tranexamic acid, or tea tree oil. Preservatives may also be included in the compositions to extend shelf life, especially for hydrophilic compositions, and fragrances and sunscreens (e.g., meradimate, oxtinoxate, octisalate, oxybenzone) may be included to enhance cosmetic and protective properties of the composition.

As another example, a texture agent that enhances the texture of the composition, enhances the comfort and feel of the composition, and eases the application of composition by the user may also be included. In some embodiments, the texture agent may include kaolin, silica, zinc stearate, polymethylsilsesquioxane, and magnesium sterate. The texture agent may have anticaking properties that prevents the cosmetic from caking. In a preferred embodiment, the texture agent may account for about 3 to 8% by weight composition of the composition.

The table below includes components of a dual use analgesic and cosmetic composition for topical application. The percentages by weight are based upon the total weight of the topical composition. The agent in question may be sourced from animal, plants, or minerals and/or may be synthetically manufactured (or any combination thereof).

| | Range | |
|---|---|---|
| | Amount | |
| | (% By | |
| Component | Weight) | Agent |
| Beeswax | 15-25 | Hydrophobic waxy |
| | | ingredient/skin |
| | | conditioning agent |
| Butyrospermum Parkii | 11-13 | Hydrophobic waxy |
| (Shea) Butter | | ingredient/skin |
| | | conditioning agent |
| Lauryl/Myristil Benzoate | 10-15 | Skin conditioning agent |
| (Corum 5014) | | |
| Hydrogenated Methyl Abietate | 8-13 | Stabilizing agent |
| Caprylic/Capric Triglyceride | 1-10 | Skin conditioning agent |
| Jojoba Esters | 3-6 | Skin conditioning agent |
| Kaolin | 3-6 | Texturizing agent |
| Phenol | 3-6 | Analgesic |
| Camphor | 3-11 | Analgesic |
| Helianthus Annus | 1-3 | Skin conditioning agent |
| (Sunflower) Seed Wax | | |
| Mica | 1-2 | Pigment base |
| Lauroyl Lysine | 1-3 | Skin conditioning agent |
| Cetearyl Alcohol | 1-3 | Stabilizer |
| Polyglyceryl-4 Isostearate | 0.5-2 | Stabilizer |
| Hexyl Laurate | 0.5-2 | Skin Conditioning Agent |
| Cetyl PEG/PPG-10/1 Dimethicone | 0.5-2 | Skin Conditioning Agent |
| Acacia Decurrens Flower Wax | 0.1-1 | Skin Conditioning Agent |
| Polyglycerin-3 | 0.1-1 | Skin Conditioning Agent |
| Titanium Dioxide | 1-10 | Pigment base |
| Yellow Iron Oxide | 1-5 | Colorant/Pigment |
| Red Iron Oxide | 0.5-4 | Colorant/Pigment |
| Black Iron Oxide | 0.1-5 | Colorant/Pigment |

The composition may take any form that allows for convenient and easy application by a user including to the lip and surrounding areas. In one preferred embodiment, the composition is substantially hydrophobic or anhydrous formed of predominantly hydrophobic or oil-based components. In other embodiments, a composition that includes hydrophilic components may be prepared and emulsifying agents are used. The cosmetic and analgesic composition may be a cream, wherein a cream is defined as an emulsion comprised of, but not limited to, oil and water in approximately equal proportions. In alternate embodiments, the composition's ingredients and ratios, may be modified such that the consistency of the composition may be a powder, liquid, gel, ointment, paste, or pressed powder.

The composition is applied to the lips and/or facial labial region currently affected by cold sores or fever blisters. It may also be used in other areas with painful lesions such as from chicken pox or shingles; itchy or irritated skin, or any other desired area needing cosmetic coverage and pain relief (any of these areas may be referred to as an “affected area”). The composition may be applied through any desired technique; by dispensing from a container or tube directly onto the affected area; by using an applicator to apply a desired quantity to the affected area; by using a flexible substrate that has a predispensed amount of the composition and then applying the composition to the affected area by moving the substrate, with composition side toward the affected area, pressing, then removing the substrate, by spraying, by manual application by a finger or tool, or any other desired technique.

In one embodiment, the user of the composition, swipes a fingertip on a few times across the surface of the product to collect a desired quantity for use, then the user gently dabs the finger upon which the compound has been transferred on and around the affected area; then the user may blend the composition by gently smoothing out the applied composition outwardly from the affected area to ensure desired coverage and to blend into the surrounding skin. The user may reapply the compound as desired to ensure continued cosmetic coverage or for additional pain relief if the analgesic effect has become diminished.

Preferably, a thick layer of the composition may be applied to the affected area. The composition should be lightly handled when applied to the skin to avoid further irritation to the affected area. The composition should cover the entire infected area, allowing a few minutes to elapse to allow the composition to dry. Once the composition is dry, additional makeup can gently be applied directly over the formulation. In that way, the composition serves as an analgesic cosmetic primer. In various embodiments tools to apply the composition are not used, or if they are, disposable applicators are used to prevent cross-contamination. The composition can be removed by makeup wipes, soap, baby oil, makeup remover, or anything used for facial cleansing.

In one embodiment, the cosmetic analgesic composition may be part of a kit including a container or housing for the cosmetic composition and at least one applicator. Examples of appropriate containers are known in the cosmetic field and include compact type cases, lip stick applicators, lip gloss containers, and the like. A user may use disposable applicators to prevent cross-contamination. The container may be made of plastic, silicon, metal, or any other suitable material. The container may be spherical, tube-shaped, circular, rectangular, square, or some other shape. In an embodiment, the container may be circular and may have a lid hingedly coupled to the base, much like a make-up compact. The base may have an indentation that mates with a corresponding engagement lip protruding from the perimeter of the lid. In this embodiment, the composition is stored inside the base and is compacted within a cavity in the base. The interior surface of the lid may have a mirror or be made of a reflective material. In an embodiment, the container may further include a carrying case for one or more disposable applicators.

The applicator may be a wand type applicator with a handle and a tip such as a lip gloss applicator or a small brush. For example, the handle portion of the wand may be made of plastic, silicon, or any suitable material. The tip may be made of any suitable material such as foam, silicon, cloth, or some other suitable material. In another embodiment, the applicator may be a finger cot. The finger cot may be applied to the finger and used to apply the cosmetic analgesic composition. The finger cot may be made of flexible plastic, silicon, cloth, or some other suitable material. The kit may include a number of single use, disposable applicators. In other embodiments, the applicator may be washable and reusable. The applicator may be infused with an anti-microbial or anti-viral material.

The composition can be removed by makeup wipes, soap, baby oil, makeup remover, or anything used for facial cleansing.

In one method of creating a preferred composition embodiment, one begins by separating the ingredients into three separate compositions, referred to herein as Premix A, Premix B, and Premix C. Generally, in a preferred method of manufacturing, the dual use cosmetic and analgesic composition is manufactured by preparing Premix A by mixing one or more cold sore analgesics with a stabilizing agent; preparing Premix B by mixing at least one waxy ingredient with at least one conditioning agent or texture agent; preparing Premix C by mixing at least one coloring agent with pigment base (and optionally adding an emollient); and creating the final composition by combining Premix B and C with Premix A and a homogenizer.

Premix A is preferably comprised of camphor and phenol. These are combined to create camphorated phenol. Once formed, the camphorated phenol is mixed with Corum at fifty-five (55) degrees Celsius to form Premix A. In a specific embodiment, Corum 5014 is made from plant-derived alkyl group and may replace compounds utilizing C12-15 Alkyl benzoate, isopropyl myristate and isopropyl palmitate. Corum 5014 acts as an emollient and dispersing agent for the inventive composition, and more generally is a solubilizer and fixative for perfume oils, an emollient and dispersing agent for antiperspirant products, and is an excellent solubilizer for UV filter agents.

In alternate embodiments, said mixing temperature may be altered to any degree understood as viable by an individual skilled in the art of cosmetic creation. Premix A includes the composition's topical cold sore analgesic. In the preferred embodiment, subsequent to the unification of all three premixes, said composition is formed. Alternative embodiments of the present invention may use additional or alternate topical pain relievers as part of Premix A. Examples are discussed herein.

In one embodiment, the camphor in the camphorated phenol comprises at least three percent (3%), but no more than eleven percent (11%) of the final composition comprising Premix A, Premix B, and Premix C. Additionally, in one embodiment, the camphorated phenol is a 1:1 mixture (by mole) and a 2:1 mixture (by mass) of camphor and phenol. Also, as no covalent interactions exist between these two molecules, the camphorated phenol cannot be one solid molecule and is, instead, a mixture of two compounds.

Premix B, in the preferred embodiment, is comprised of beeswax, shea butter, fractionated coconut oil, and ACTICIRE® (a texturizing agent). The above-mentioned sub-components of Premix B are combined and then mixed at a desired temperature range, preferably greater than seventy-five (75) degrees Celsius, but less than eighty (80) degrees Celsius, with Meristant 101 (hydrogenated methyl abietate), a solubilizer, which helps to increase water resistance, and with cetearyl alcohol, an emulsifier or otherwise a stabilizing agent. In alternate embodiments, said mixing temperature may be altered to any degree understood as viable by an individual skilled in the art of cosmetic creation.

Premix C, a cosmetic component of the analgesic cosmetic compound, is comprised of colorants or pigments combined with AMIHOPE® LL. AMIHOPE® LL is an amino-acid derivative from Ajinomoto Inc., which is made from L-Lysine and fatty acids. AMIHOPE® LL imparts a soft and silky feel to the skin as well as excellent emolliency, and also can relieve drying feel of inorganic powders. The AMIHOPER LL particles have flat and hexagonal shape, resulting in excellent adherence to skin which provides long wearing properties. As L-Lysine is believed by some practitioners to be of assistance in treating cold sores, Premix C may also provide a therapeutic aspect to the affected area when applied as part of the inventive compound.

In one embodiment, subsequent to the creation of all three premixes, Premix B is combined with Premix C. Thereafter, said B and C mixture is combined with Premix A and a homogenizer, which ensures that the mixture remains even throughout. Throughout said combination procedure, the temperature, in the preferred embodiment, is fifty-five (55) degrees Celsius. In alternate embodiments, said mixing temperature may be altered to any degree understood as viable by an individual skilled in the art of cosmetic creation. When composed as described, the mixture of Premix A, B, and C forms an embodiment of the preferred compound of the present invention.

The particular implementations shown and described above are illustrative of the invention and are not intended to otherwise limit the scope of the present invention in any way. Indeed, for the sake of brevity, conventional aspects of the invention may not be described in detail. Methods may include more, fewer, or other steps. Additionally, steps may be performed in any suitable order without departing from the scope of the invention. Many alternative or additional functional relationships or physical connections may be present in a practical system.

Changes and modifications may be made to the disclosed embodiments without departing from the scope of the present invention. These and other changes or modifications are intended to be included within the scope of the present invention, as expressed in the following claims, which illustrate non-limiting embodiments of the present invention.

What is claimed is:

1. A dual use cosmetic and analgesic composition for topical application to the lip or labial region comprising: at least one cold sore analgesic, at least one pigment, at least one waxy ingredient; and at least one stabilizing agent, wherein the composition is substantially hydrophobic.

2. The composition of claim 1 wherein the cold sore analgesic is a pain reliever for treatment of cold sores.

3. The composition of claim 1 further comprising at least one pigment base.

4. The composition of claim 2 further comprising at least one texture agent.

5. The composition of claim 2 further comprising at least one conditioning agent.

6. The composition of claim 1, wherein the at least one cold sore analgesic comprises camphor.

7. The composition of claim 6, wherein the cold sore analgesic is camphorated phenol.

8. The composition of claim 7, wherein the camphorated phenol is present in the composition in amount of about 3 to 11% by weight of the composition.

9. The composition of claim 1, wherein the stabilizing agent is cetearyl alcohol.

10. The composition of claim 1, wherein the composition further comprises a stabilizing agent that is a solubilizer and the solubilizer is hydrogenated methyl abietate.

11. The composition of claim 1, wherein the pigment comprises iron oxide.

12. The composition of claim 3, wherein the pigment base comprises titanium dioxide.

13. The composition of claim 1 wherein the pigment comprises iron oxide and is present in the composition in amount between about 0.1 to 5% by weight.

14. The composition of claim 3 wherein the pigment and pigment base together comprise about 1 to 10% by weight of the composition.

15. The composition of claim 1 wherein the dual use cosmetic and analgesic composition is water proof.

16. A cosmetic application apparatus comprising a container comprising the dual use cosmetic and analgesic composition of claim 1 and at least one applicator for topical application of the composition.

17. A dual use cosmetic and analgesic composition for topical application to the lip or labial region comprising: at least one cold sore analgesic, at least one pigment, at least one waxy ingredient, and at least one emulsifying agent,
wherein the cold sore analgesic is substantially hydrophilic.

18. A kit comprising
a container comprising a housing containing a dual use cosmetic and analgesic composition, and
at least one applicator for topical application of the composition,
wherein the composition comprises at least one cold sore analgesic, at least one pigment, at least one pigment base, at least one waxy ingredient, and at least one stabilizing agent.

19. The kit of claim 18 wherein there are a plurality of applicators for topical application of the composition.

20. A method of manufacturing a dual use cosmetic and analgesic composition for topical application comprising
preparing a first premix by mixing a cold sore analgesic with a stabilizing agent;
preparing a second premix by mixing at least one waxy ingredient with another ingredient;
preparing a third premix comprising a pigment;
preparing a fourth premix by mixing the second premix and third premix; and
combining the fourth premix with the first premix and a homogenizer.

21. The method of claim 20 wherein the first premix is prepared at a temperature of about above 45 C.

22. The method of claim 20 wherein the step of combining the fourth premix with the first premix and homogenizer is prepared at a temperature of about above 40 C.
